(19) United States
(12) Patent Application Publication
(10) Pub. No.: US 2010/0087924 A1
(43) Pub. Date: Apr. 8, 2010

(54) Expandable Intervertebral Prosthesis Device for Posterior Implantation and Related Method Thereof

(75) Inventor: Vincent Arlet, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/520,334

(22) PCT Filed: Jan. 7, 2008
(86) PCT No.: PCT/US08/50383

§ 371 Date: Jun. 19, 2009

Related U.S. Application Data
(60) Provisional application No. 60/878,894, filed on Jan. 5, 2007.
(60) Provisional application No. 60/904,502, filed on Mar. 2, 2007.

(51) Int. Cl. A61F 2/44; A61B 17/58

(52) U.S. Cl. 623/17.12; 606/93; 606/86 R

(57) ABSTRACT

A method and device for the insertion of a vertebral cage that may be done through a posterior approach that is minimally traumatic and does not require retraction of the spinal cord, dural sac or spinal nerves. The insertion of the cage may therefore be parallel to the spinal cord and the cage will be rotated ninety degrees (or as desired or required) in the vertebral body defect or as applicable to achieve its proper positioning before expansion.

### CROSS REFERENCE TO RELATED APPLICATIONS

This application claims priority from U.S. Provisional Patent Application Ser. No. 60/878,894, filed on Jan. 5, 2007, entitled “Cement-filled Cage for Use in the Treatment of Spinal Tumors” and U.S. Provisional Application Ser. No. 60/904,502, filed Mar. 2, 2007 entitled “Cement-filled Cage for Use in the Treatment of Spinal Tumors,” the entire disclosures of which are hereby incorporated by reference herein in their entirety.

### BACKGROUND OF THE INVENTION

Reconstruction of the anterior spine column may be necessary in conditions such as, but not limited thereto, fracture of the spine, tumors or infections of the spine. Conventional expandable cages for vertebral body replacement have been described to be inserted from the front of the spine. The conventional devices rely on, for example, a sort of mechanical device (ratchet type device or threaded expansion screw device) to achieve the expansion of the cage. Theses conventional cages are bulky and cannot be inserted from the back without an extended posterior or posterolateral approach to the spine and very specialized access methods that can be deemed dangerous by some surgeons.

Previous methods of insertion of such mechanical cages from the back are associated with the maximum invasive nature of anterior approach—See: 1) Spine J. 2007 Jun. 21, “The Use of an Expandable Cage for Corpectomy Reconstruction of Vertebral Body Tumors through a Posterior Extracavitary Approach: A Multicenter Consecutive Case Series of Prospectively Followed Patients,” Shen F H, Marks I, Shaffrey C, Ouellet J, Arlet V, and 2) J Neurosurg Spine. 2006 September; 5(3), “Expandable Cage Placement via a Posterolateral Approach in Lumbar Spine Reconstructions,” Technical note, Hunt T, Shen F H, Arlet V, of which both of these disclosures are hereby incorporated by reference herein in their entirety.

Insertion of such conventional mechanical and bulky expandable cages from the back require manipulation of the spinal cord and nerves, often a costotransversectomy and difficult manipulation to position the cage in place, and many different tricks to achieve the expansion of the cage.

An aspect of the present invention device and method provides a new concept of low profile expandable cage that could be used for posterior insertion and through a minimal invasive approach that does not require any manipulation of the dural sac or the spinal nerves.

### SUMMARY OF THE INVENTION

Some of the exemplary aspects of the present invention low profile expandable cage provide, but not limited thereto, the following features and advantages:
The insertion of the cage may be done through a posterior approach that is minimally traumatic and does not require retraction of the spinal cord, dural sac or spinal nerves. The insertion of the cage may therefore be parallel to the spinal cord and the cage will be rotated 90 degrees (or as desired or required) in the vertebral body defect to achieve its proper positioning before expansion.
The cage is low profile with a collapsed height of less than about 20 mm (or height as desired or required), and a foot print that will match the level to be inserted (e.g., thoracic or lumbar spine).
The expansion of the cage is achieved through injection of cement or any liquid or paste like substance that will harden with time or with increased temperature (e.g., human body temperature or temperature as desired or required).
The cage may include two endplates that are connected through a cage body that may be expandable tubing or chamber (or the like) to accept the cement or filler material. Cage and cage body may be made of plastic or other desirable material as desired or required. The cage body tubing or chamber (or the like) can be folded in an accordion or bellows like pattern. The cage can be made of two endplates connected with cylinders (stack of cylinders) that slide and allow expansion during cement injection such as a telescopic-like or a wedding cake design in the expanded stage. The end plates can be made of titanium or other biocompatible materials like polyether ether Keton (PEEK); or other materials as desired or required.
The expansion chambers of such cage bodies may be emptied in their collapsed stage so that the injection of cement does not lead to any air or bubble during expansion that could make the construction more fragile.
The guide (e.g., driver element) to manipulate and insert the cage may be fixed to an end plate and may be used later as the “hose to inject the cement” under pressure. A locking mechanism of the guide to the cage prevents its loosening in clockwise or counter clockwise rotation. In an approach the guide (i.e., driver element) may be fixed to the cage body without an endplate. Alternatively, the driver element may be in communication with both the cage body and endplate(s).

Such new expandable cages (e.g., accordion/bellows type or stack of cylinders, or as applicable according to the present invention) may have an expansion capacity far greater than a conventional mechanical cage, whereby the expansion of such conventional cage cannot reach beyond twice the overall height of the cage in its collapsed phase. For instance, the present invention expandable cages could be at least twice, three times, or greater than a conventional mechanical cage. The magnitude of expansion can depend on, for example, but not limited thereto, the number of stacked cylinders and pleated accordions or bellows. The magnitude of expansion can depend on, for example, but not limited thereto, the thickness/height of stacked cylinders and pleated accordions or bellows, or any applicable components of the cage and/or cage body.

Due to the minimal height of the cage in its collapsed phase of the present invention, no dangerous manipulation of the dural sac is necessary.

The manipulation of the present invention cage into the vertebral body defect does not require a bulky cage holder that is always dangerous for the spinal cord

The expansion of the present invention cage can be accomplished using pragmatic, convenient and straight forward pressurization type mechanism.

An aspect of an embodiment of the present invention comprises an intervertebral prosthesis device for posterior implantation between adjacent vertebrae of a subject. The device comprising: a cage body, whereby the cage body has a lower end and an upper end defining a vertical height there between. The cage body has a transverse width and transverse length. The cage body is flexible and expandable, wherein the cage body is adapted to: change from a non-deployed state defining the vertical height being in a non-deployed vertical height (NVH) and expanded to a deployed state when filled with a filler material, defining the vertical height being in a deployed vertical height (DVH). The non-deployed vertical height cage body may be less than or equal to the transverse height or transverse length of the cage body. The device further comprises: a driver element in communication with the cage body. The cage driver element is adapted to: insert the cage from the posterior of a subject and rotate the cage between the adjacent vertebrae, and expand the cage body with the filler material.

An aspect of an embodiment of the present invention comprises a method for implanting an intervertebral prosthesis device between adjacent vertebrae from the posterior of a subject. The method comprising: inserting a cage body from the posterior of the subject into space defined between the adjacent vertebrae; rotating the cage body between the adjacent vertebrae, and expanding the cage body with the filler material to fill a space defined between the adjacent vertebrae.

These and other objects, along with advantages and features of the invention disclosed herein, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 illustrates a schematic posterior view of a spine of a subject.

FIGS. 2A-B illustrate a schematic side view and posterior view of the spine, respectively.

FIGS. 3A-B illustrate a schematic plan view (axial overhead view) and side (lateral or elevation view) of a vertebra, respectively.

FIG. 4 illustrates a schematic plan view (axial overhead view) of a vertebra.

FIG. 5 is a schematic cross section of the human torso through the first lumbar vertebra showing the spinal cord along with related anatomy.

FIG. 6 is a schematic side view of illustrating that the spinal canal or vertebral foramen of the vertebra 13 is generally circular and smaller than a ring finger.

FIG. 7 illustrates a schematic side view of a portion of the spine or spinal column with a portion removed there from.

FIGS. 8A-C illustrates a schematic cage in its collapsed or non-deployed state in the elevation view, plan (overhead) view, and perspective view, respectively.

FIG. 8D illustrates the schematic perspective view of the cage of FIG. 8C in its expanded or deployed state.

FIG. 9A is a schematic transverse cross section of the human torso through the second thoracic vertebra showing the spinal cord and vertebral foramen along with related anatomy.

FIG. 9B is an enlarged partial view of the human torso and vertebra illustrated in FIG. 9A.

FIGS. 10A-D are schematic views of the vertebra or area vacated by all or part of the vertebra as shown in FIG. 9B illustrating progressive stages of the cage being oriented and manipulated from the posterior approach.

FIGS. 11A-B illustrate schematic side views of a portion of the spine or spinal column with a portion removed there from with cage oriented accordingly, in the collapsed or non-deployed state and expanded or deployed state, respectively.

FIGS. 12A-C illustrate schematic side views of a portion of the spine or spinal column with a portion removed there from with cage oriented accordingly, in the following stages: collapsed or non-deployed state, partially expanded or partially deployed state, and fully expanded or deployed state, respectively.

FIGS. 13A-B illustrate schematic side views of a portion of the spine or spinal column with Kyphosis with a portion removed there from with cage oriented accordingly, in the following stages: partially expanded or partially deployed state, and fully expanded or deployed state, respectively.

FIG. 14 schematically illustrates the cage in a fully expanded or deployed state using the driver element and its related components, and whereby the driver element is in communication with the cage body.

FIG. 15 schematically illustrates the cage 51 in a fully expanded or deployed state using the driver element and its related components, and whereby cage rods are implemented as part of the cage construction.

FIGS. 16A-C schematically illustrate the cage having a cage body of the accordion type in the collapsed or non-deployed state, partially expanded or deployed state, and in the fully expanded or deployed state, respectively.

FIGS. 17A-C schematically illustrate the cage having a cage body of the telescopic type (wedding cake) in the following stages: collapsed or non-deployed state, partially expanded or deployed state, and fully expanded or deployed state, respectively.

### DETAILED DESCRIPTION OF THE INVENTION

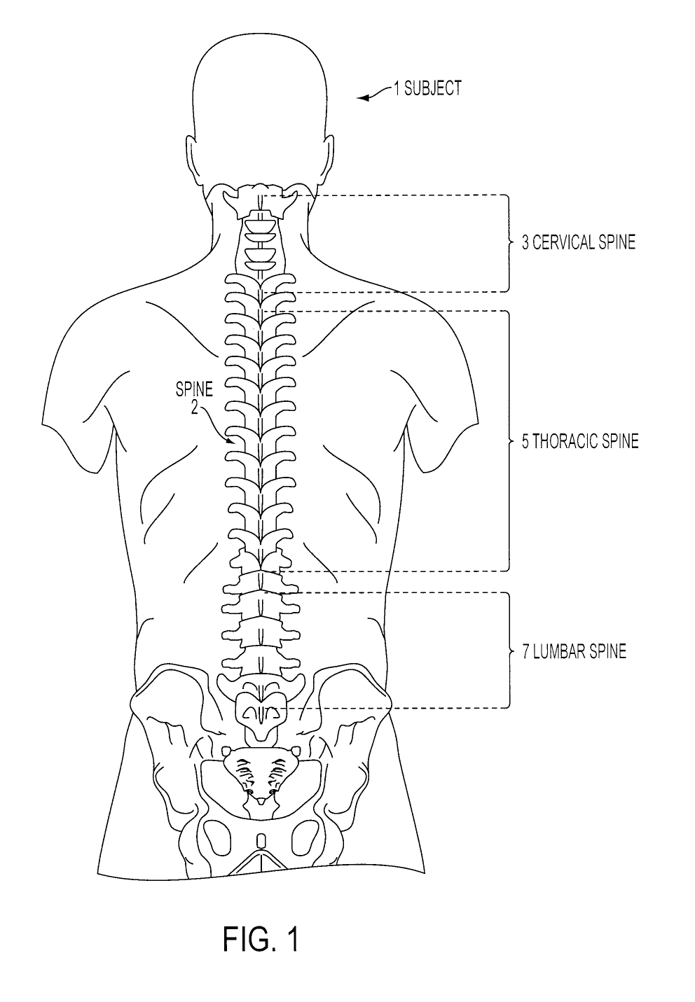

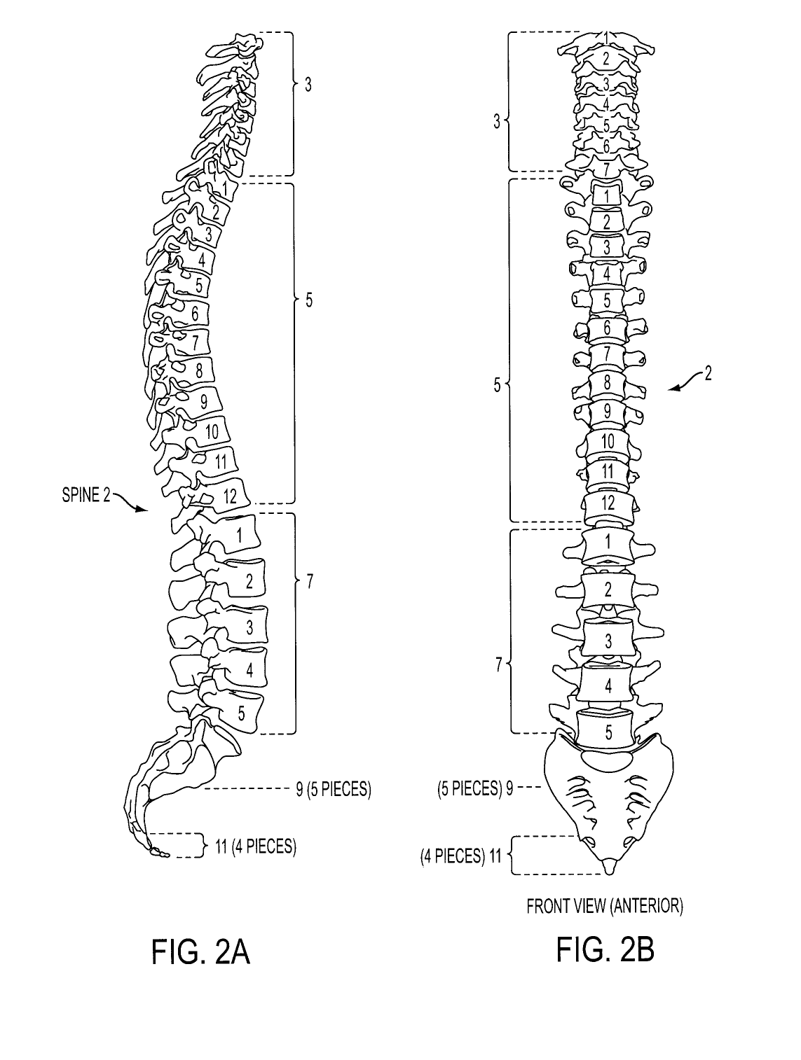

FIG. 1 illustrates a schematic posterior view of a spine 2 of a subject 1 and FIGS. 2A-B illustrate a schematic side view and posterior view of the spine 2, respectively. The normal anatomy of the spine of a human 1 is usually described by dividing up the spine 2 into three major sections: the cervical vertebrae 3, the thoracic vertebrae 5, and the lumbar vertebrae 7. Below the lumbar vertebrae 7 is a bone called the sacrum 9 and the coccyx 11, which is part of the pelvis. Each section is made up of individual bones called vertebrae 13. There are seven cervical vertebrae, twelve thoracic vertebrae, and five lumbar vertebrae.

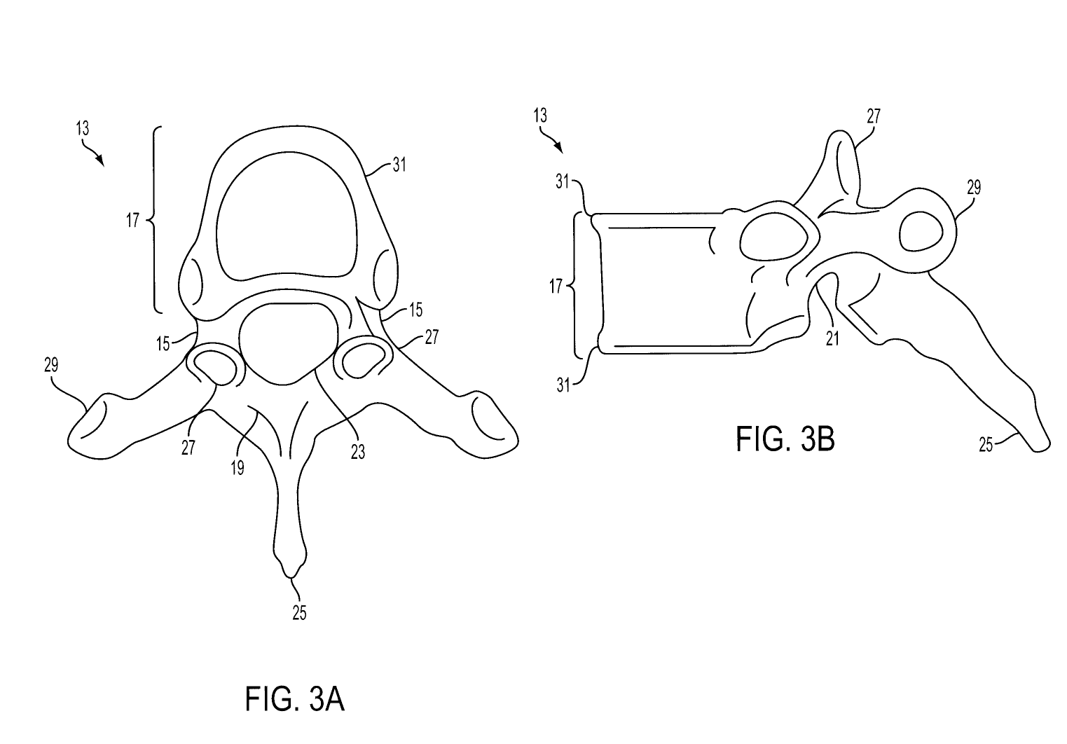

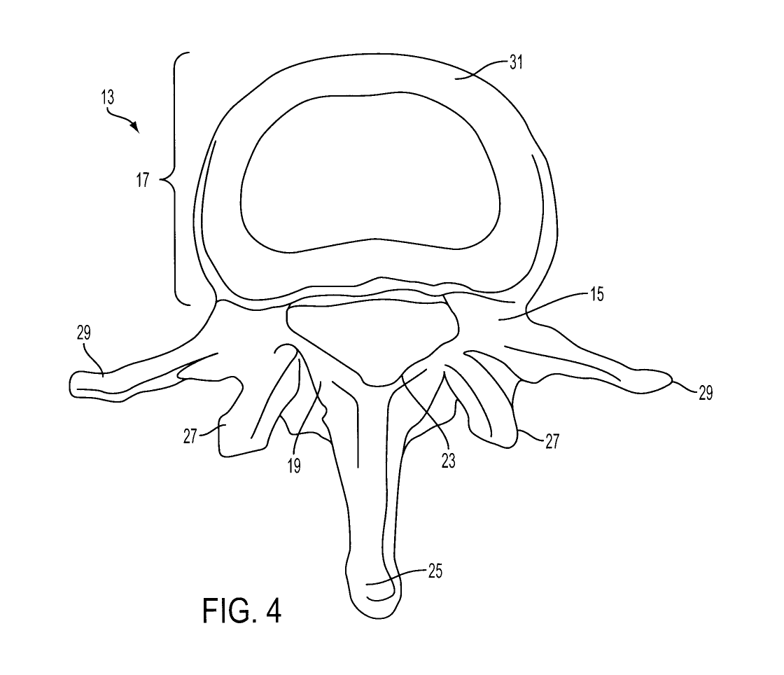

FIGS. 3A-B illustrate a schematic plan view (axial overhead view) and side (lateral or elevation view) of a vertebra 13, respectively. FIG. 4 illustrates a schematic plan view (axial overhead view) of a vertebra 13. An individual vertebra 13 is made up of several parts. The vertebra consists of two stout rounded pedicles 15, one on each side which spring from the body 17 and which are united posteriorly by two flat plates or laminae 19. A small notch is located above (not shown) and a small notch 21 is located below the pedicle 15 (called the superior and inferior vertebral notches, respectively). The vertebral foramen 23 (section of the spinal canal) is small, and of a circular form that accommodates the spinal cord (not shown) that vertically (axially) transverse through it. The spinous process 25 is long, triangular on coronal section, directed obliquely downward. The superior articular processes 27 are thin plates of bone projecting upward from the junctions of the pedicles and laminae 19. The transverse processes 29 arise from the arch behind the superior articular processes 27 and pedicles 15; they are thick, strong, and of considerable length, directed obliquely backward and lateralward, and each ends in a clubbed extremity, on the front of which is a small, concave surface, for articulation with the tubercle of a rib (not shown). The vertebral body 17 is a thin ring of dense cortical bone. The vertebral body is generally shaped like an hourglass, thinner in the center with thicker ends. Outer cortical bone extends above and below the superior and inferior ends of the vertebrae 13 to form rims or cortical rims 31. The superior and inferior endplates are contained within these rims of bone. The body 17 is composed of cancellous tissue, covered by a thin coating of compact bone; the latter is perforated by numerous orifices, some of large size for the passage of vessels; the interior of the bone is traversed by one or two large canals, for the reception of veins, which converge toward a single large, irregular aperture, or several small apertures, at the posterior part of the body.

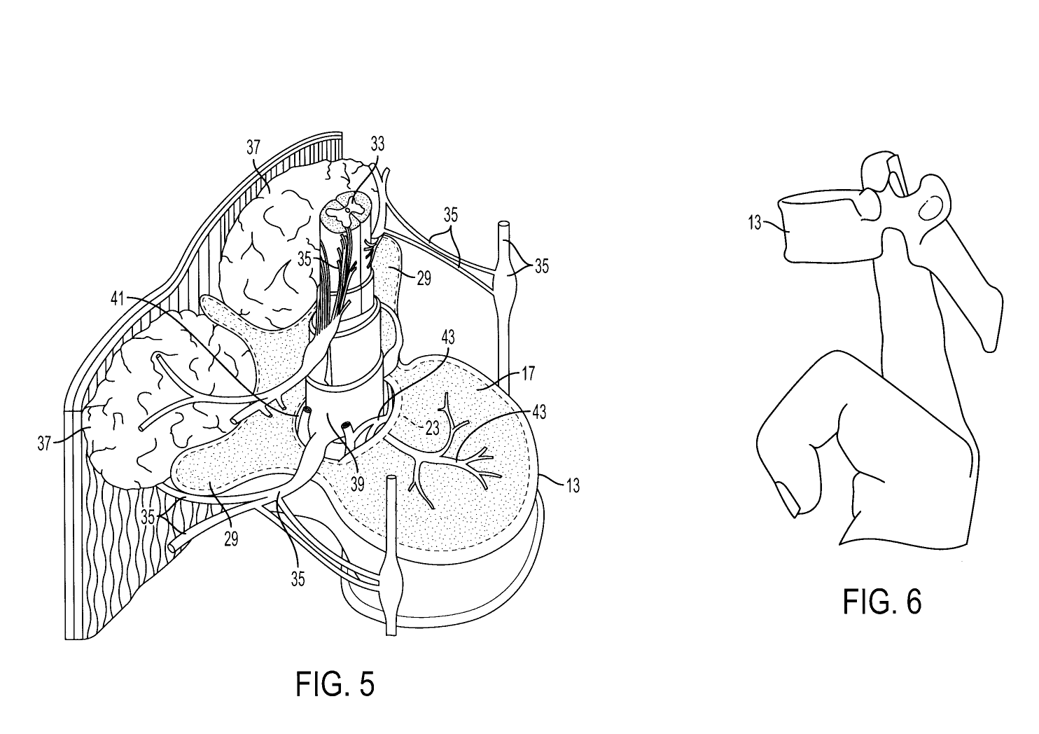

FIG. 5 is a schematic cross section of the human torso through the first lumbar vertebra showing the spinal cord 33 along with related anatomy such as 43 vasculature, 41 epidural space, 39 dura matter, vertebral muscles 37, spinal nerves 35, transverse process 29, spinous process 25, vertebral foramen 23, and body 17 of the vertebra 13.

Referring generally to FIG. 5, the spinal cord 33 is part of the central nervous system of the human body. It is a vital pathway that conducts electrical signals from the brain to the rest of the body through individual nerve fibers 35. The spinal cord 33 is a very delicate structure that is derived from the ectodermal neural groove, which eventually closes to form a tube during fetal development. From this neural tube, the entire central nervous system, our brain and spinal cord, eventually develops. Up to the third month of fetal life, the spinal cord is about the same length as the canal. After the third month of development, the growth of the canal outpaces that of the cord. In an adult the lower end of the spinal cord usually ends at approximately the first lumbar vertebra, where it divides into many individual nerve roots (L1).

Still referring generally to FIG. 5, the spinal canal or vertebral foramina 23 is the anatomic casing for the spinal cord. The bones and ligaments of the spinal column or spine 2 are aligned in such a way to create a canal or vertebral foramina 23 that provides protection and support for the spinal cord. Several different membranes enclose and nourish the spinal cord and surround the spinal cord itself. The outermost layer is called the “dura mater” or “dura sac” 39. The dura is a thin membrane that encloses the brain and spinal cord and prevents cerebrospinal fluid from leaking out from the central nervous system. The space between the dura and the spinal canal is called the “epidural space” 41. This space is filled with tissue, vessels and large veins (various vasculature 43). The epidural space is important in the treatment of low-back pain, because it is into this space that medications such as anesthetics and steroids are injected in order to alleviate pain and inflammation of the nerve roots.

FIG. 6 is a schematic side view of illustrating that the spinal canal or vertebral foramen of the vertebra 13 is generally circular and smaller than a ring finger, and becoming triangular toward the cervical and lumbar ends.

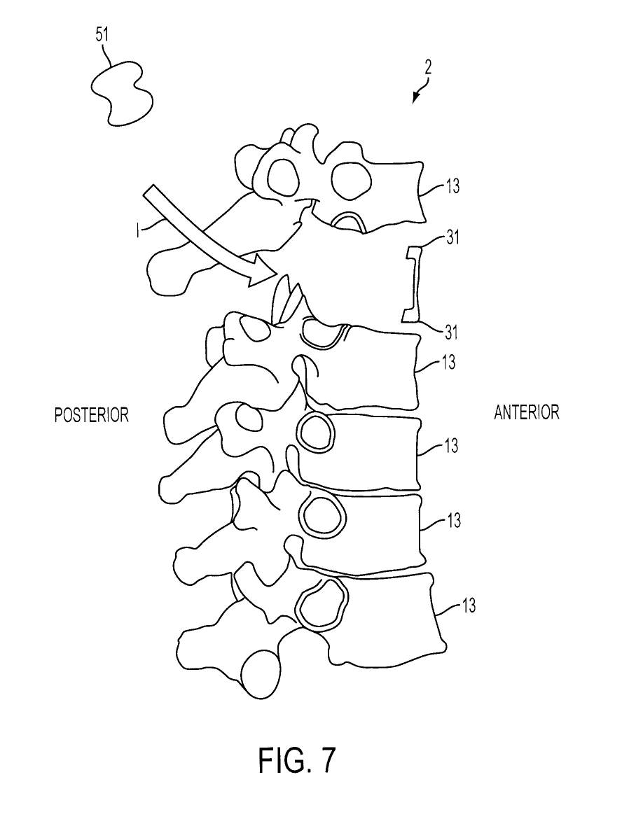

FIG. 7 illustrates a schematic side view of a portion of the spine 2 or spinal column with a portion removed there from. For instance, as illustrated, most of the vertebral body has been removed from the vertebra second from the top as illustrated, except for the anterior cortical rims 31. For instance, in an approach when the vertebral body is removed from the back prior to placement of the expandable cage, the superior and inferior end plate of the vertebral body may be removed as well. For example, the anterior part of the endplate can be left in place. However most of the superior and inferior part of the endplate of the vertebral body (e.g., involved with the tumor has to be removed) so the cage can expand to the superior endplate of the adjacent inferior vertebra, and to the inferior endplate of the adjacent superior vertebra. Next, in accordance with the present invention device and related method an expandable cage 51 is inserted as desired and required (arrow “I”) into the space or area of the vacated by the removed vertebra or portion thereof and ultimately into the proper location, position and alignment with the vertebrae without damaging or severing the spinal cord (not shown).

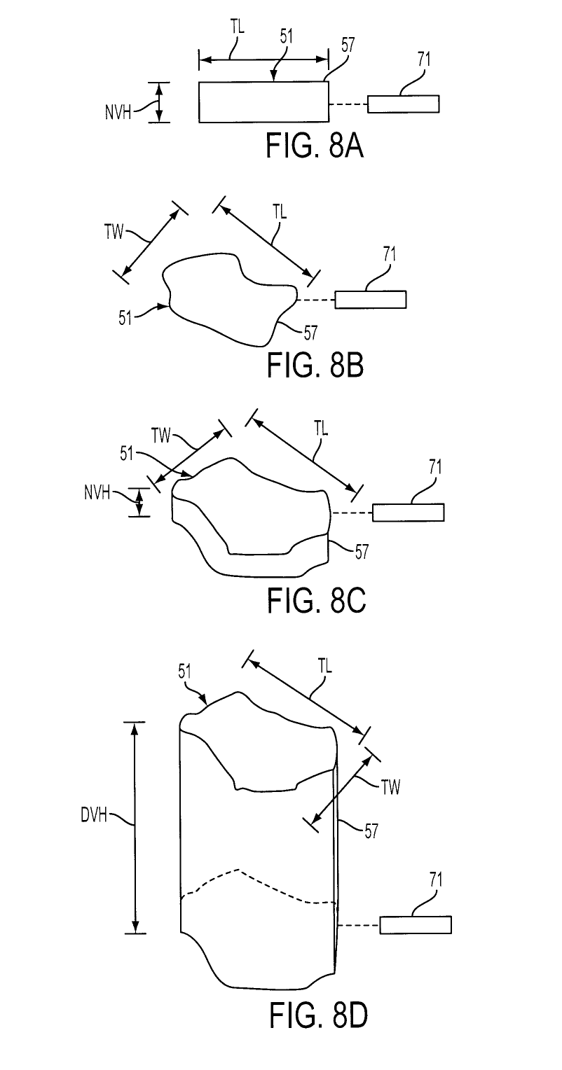

Referring to FIGS. 8A-C, the cage body 57 of the cage 51 is schematically captured in its collapsed or non-deployed state having a non-deployed vertical height (NVH) and a transverse width (TW) and transverse length (TL), in the elevation, plan (overhead), and perspective views, respectively. Referring to FIG. 8D, the cage body 57 of the cage 51 is shown in its expanded or deployed state having a deployed vertical height (DVH) and a transverse width (TW) and transverse length (TL). The non-deployed vertical height (NVH) is less than either the transverse width (TW) or transverse length (TL). Alternatively, the non-deployed vertical height (NVH) is less than each of the transverse width (TW) and transverse length (TL). Some typical cross-section shapes used would be circular, kidney shaped, or any geometrical shape as desired or required for fit to anatomy or surgical procedure.

In an embodiment, for the cage 51 to be inserted safely from the back without touching or retracting the spinal cord the cage 51 must be in its collapsed stage (non-deployed state) not any taller (vertically) than about 15-20 mm maximum (i.e., non-deployed vertical height (NVH)), or as desired or required. Its cross section in the lateral or horizontal direction (i.e., the transverse width (TW) and transverse length (TL) may vary depending of the location to be inserted as the cross section of the vertebral bodies varies from the thoracic or lumbar spine. A lateral or horizontal cross section between about 25-30 mm may be used depending of the level, or as desired or required. It is essential to understand that such low profile expandable cage make their insertion safer as there is no need to retract the spinal cord and rotation and expansion of the cage will then allow its perfect placement. A driver element 71 is in communication with the cage. The driver element 71 may be adapted to position and orient the cage and/or fill the cage with a filler material such as cement or the like. Other filler materials may also include biologic resin that hardens after time or at body temperature, a synthetic bioactive paste that hardens with time or at body temperature. The cage 51 in its expanded or deployed state may have a deployed vertical height (DVH) of about 45-60 mm, or as desired or required.

It should be appreciated that various sizes, dimensions, contours, rigidity, shapes, flexibility and materials of any of the embodiments discussed throughout may be varied and utilized as desired or required.

It should be appreciated that while the expansion illustrated in the various embodiments discussed through out focuses on vertical or axial expansion, it should be appreciated that expansion may also be implemented in the lateral or horizontal (e.g., transverse) direction.

In an exemplary embodiment, the driver 71 may be connected to the end plate (not shown) of the cage 51 so it locks, as opposed to a non-lock in screw in mechanism that would allow it to loosen up during the rotation, for instance of the cage counterclockwise. The cage end plate (not shown) where the driver is connected may be thicker than the opposite end plate (not shown) to allow fitting of the valve (not shown) and locking mechanism (not shown) and cement insertion mechanism under pressure.

In an exemplary embodiment, the cage 51 itself may have a flexible cage body 57 that is a flexible, malleable and expandable chamber. A flexible and expandable chamber may comprise, but not limited thereto, the following structures: tube, balloon, hose, cylinder, accordion like-structure, bellows, case, shell, enclosure, sleeve, or repository. In an approach, the flexible cage body in its collapsed stage (non-deployed stage), may be under negative pressure. The negative pressure will allow the cement to have a uniform filling of the cage body avoiding air to be trapped inside the cage (e.g., bubble in cement or filing) that could cause less biomechanic resistance. As the cage is expanded a positive pressure of filler material enters and expands the cage body. The expansion of the cage is driven by the filler material. In accordance with the present invention device and related method an expandable cage 51 is inserted the space or area of the vacated by the removed vertebra or portion thereof (i.e., between adjacent vertebrae) and ultimately into the proper location, position and alignment with the adjacent vertebrae without damaging or severing the spinal cord (not shown). The cage body may be expanded in accordance with the present invention and, for example, restore the carpectomy defect.

It should be appreciated that any pressure or regulation of pressure of air or filler material may vary as desired or required. Regulation may entail, for example, at least one of the following: prevention, adjustment, reduction, amplification, or control for the flow of or quantity of air, filler material or any medium as desired or required.

It should be appreciated that the cage body 57 and related cage components discussed herein may take on all shapes along the entire continual geometric spectrum of manipulation of x, y and z planes to provide and meet the anatomical, maneuverability, safety and structural demands and requirements. Size and shape of the cage body 57 during the various stages of deployment (non-deployed, partially deployed, and fully deployed, for example) could also be manipulated by varying the compliance of the cage body walls, cage and cage body structure and inflation/expansion pressure.

Alternatively, referring to the cage body 57, the flexible and expandable chamber may comprise a structure comprising a series of cylinders in telescopic arrangement.

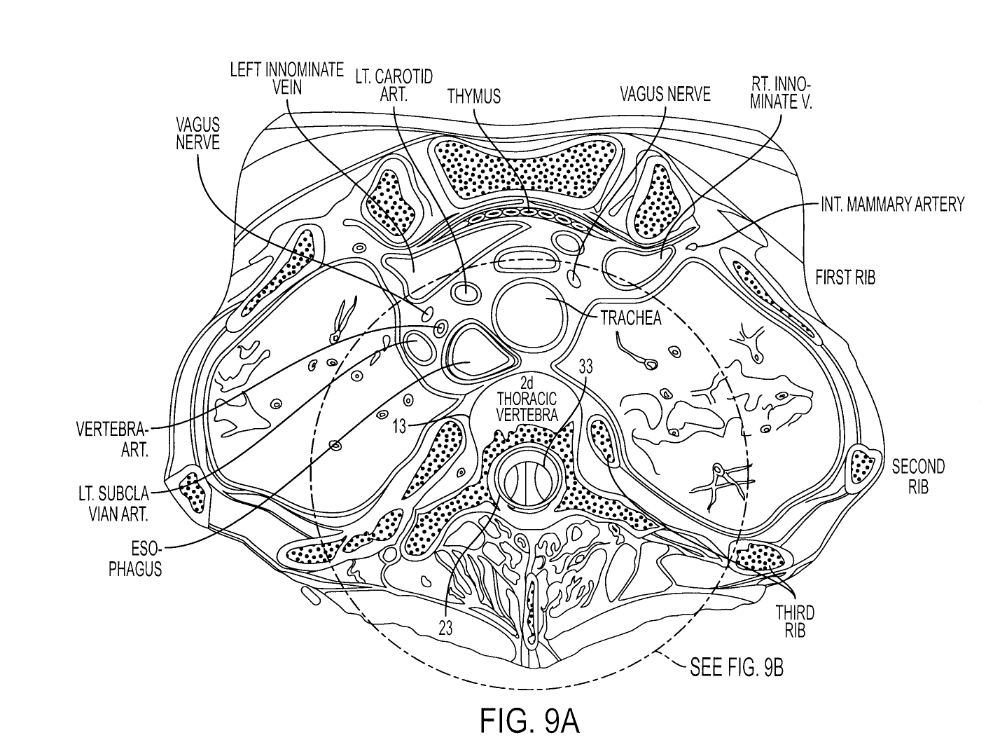

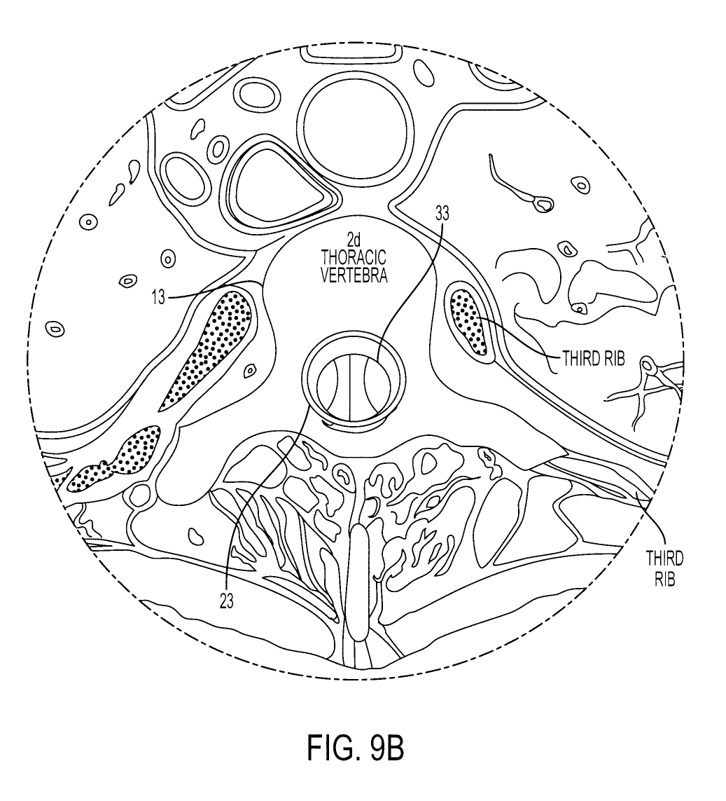

FIG. 9A is a schematic transverse cross section of the human torso through the second thoracic vertebra 13 showing the spinal cord 33 and vertebral foramen 23 along with related anatomy. FIG. 9B is an enlarged partial view of the human torso and vertebra 13 as illustrated in FIG. 9A.

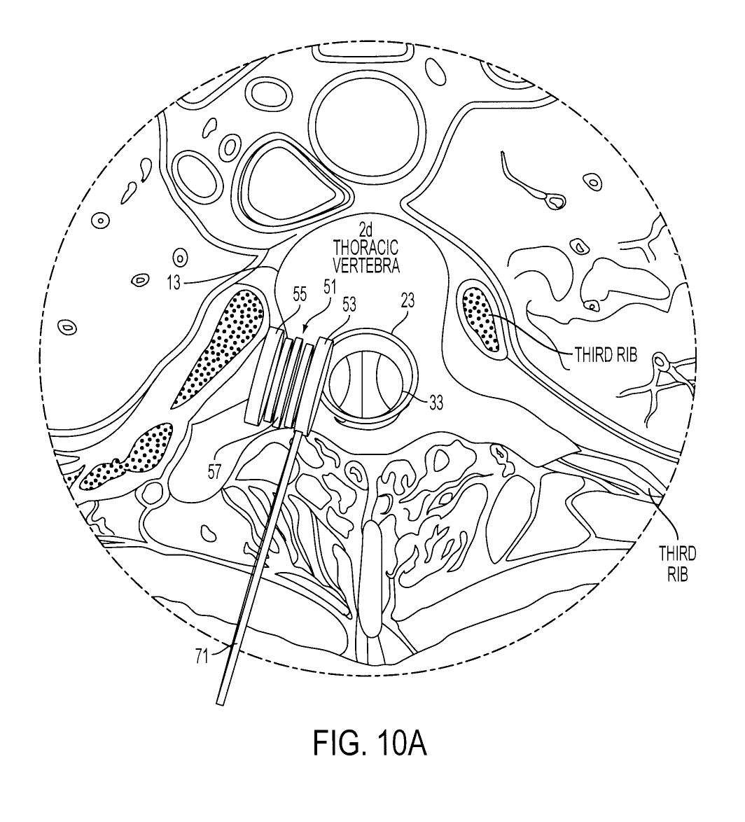

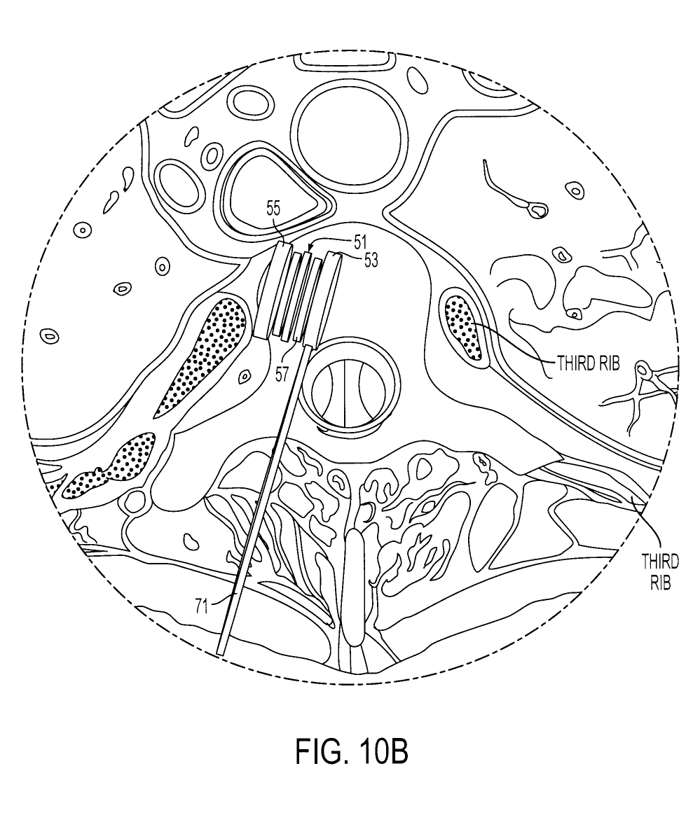

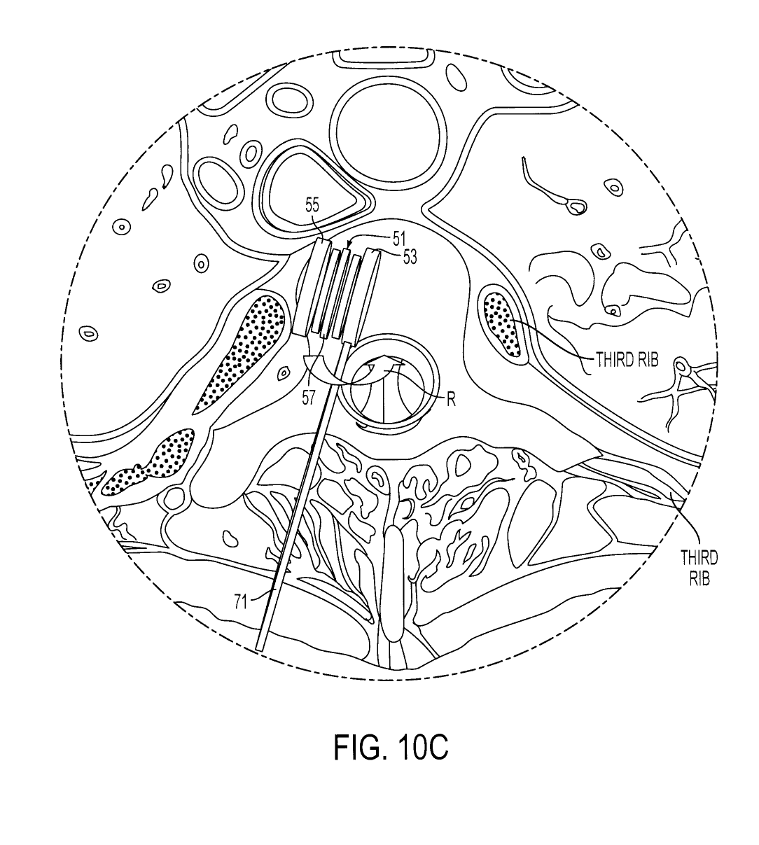

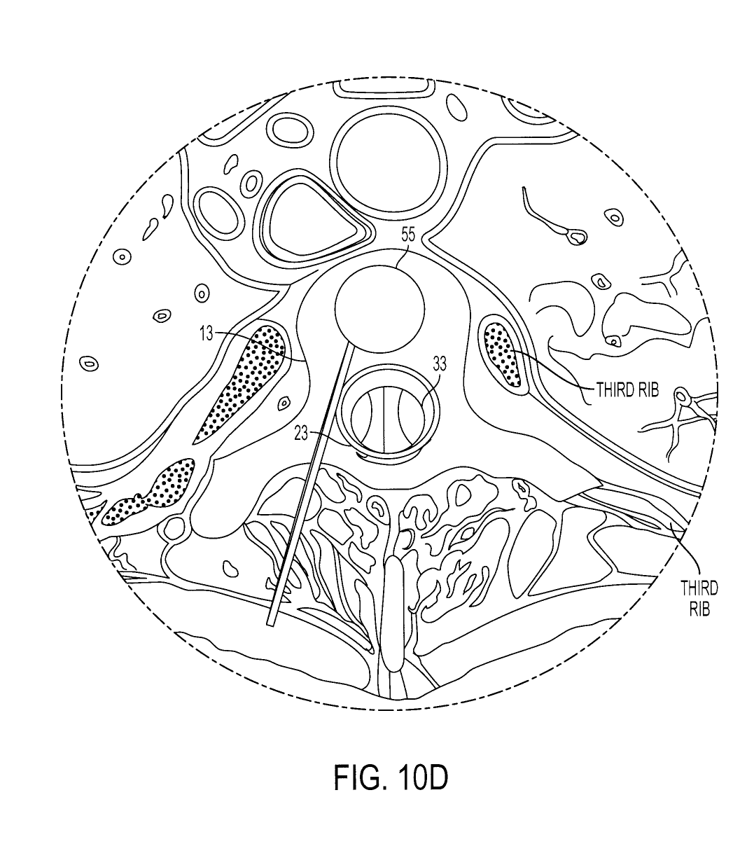

FIGS. 10A-D are schematic views of the vertebra or area vacated by all or part of the vertebra as shown in FIG. 9B illustrating progressive stages of the cage 51 being inserted, rotated and located in place by the driver element 71 into the spine of the subject as desired or required using the low profile and reduced invasiveness posterior approach of the present invention device and method. In an approach, the cage 51 has a body 57 in communication with a lower endplate 53 and upper endplate 55. As shown in FIG. 10A, the cage 51 has been at least partially inserted and manipulated with the driver element 71 from the posterior. As shown in FIG. 10B, the cage 51 has been further advanced by the driver element 71 from the posterior. As shown in FIG. 10C, the cage 51 is capable for rotation as indicated by arrow “R”. As shown in FIG. 10D, the cage 51 has been rotated and positioned into the area vacated by the vertebra. It should be appreciated that the lower endplate and upper endplate may be interchangeable and are described as upper and lower for illustration purposes only. It should be appreciated that the driver element may comprise more than one instrument or component as desired or required for the procedure.

It should be appreciated that the lower endplate and upper end plate may be a variety of structures such as, but not limited thereto, the following; housing, plate, substrate, seat, platform, pedestal, chamber, holder, case, box, base, flange, collar, panel, partition, wall or the like, or any combination thereof.

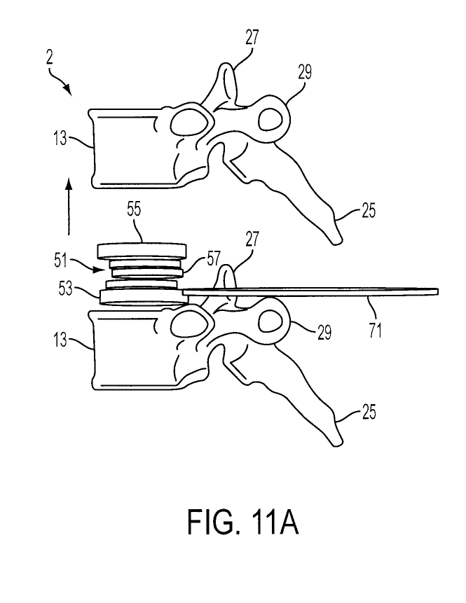

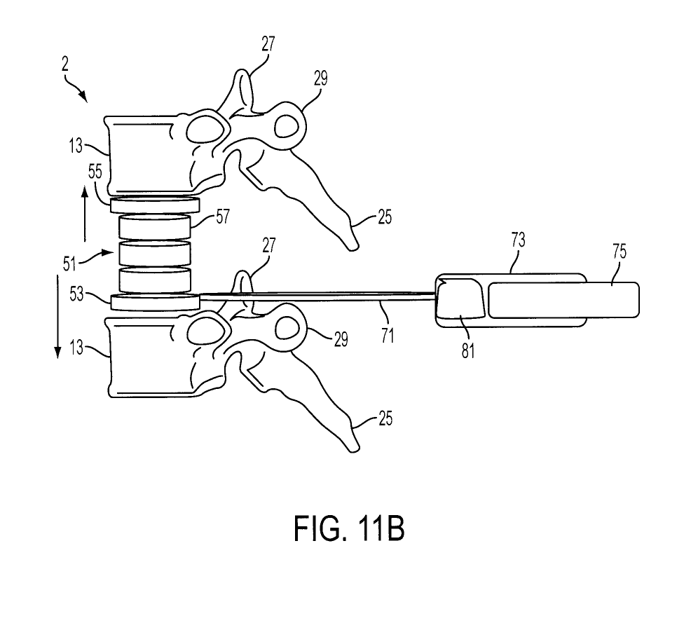

FIGS. 11A-B illustrate schematic side views of a portion of the spine 2 or spinal column with a portion removed there from. For instance, as illustrated, all of a vertebral body may be removed (or a portion of a vertebral body). An individual vertebra 13 is made up of several parts, such as the spinous process 25, superior articular processes 27, and transverse processes 29. FIG. 11A illustrates the cage 51 placed and rotated accordingly by the driver element 71 into the spine 2 having utilized the present invention posterior approach while in a collapsed or non-deployed state. FIG. 11B illustrates the cage 51 expanded or deployed state using the driver element 71 and its related components. Some related components may be the driver mechanism 73 that is adapted to deliver the cement 81 or filler material into the cage body 57 of the cage 51. In an approach, the driver 71 is adapted to deliver the cement or filler material under negative pressure. The driver mechanism 73 of the driver element 71 may comprise an actuator 75 such as a valve or piston to advance the cement or filler.

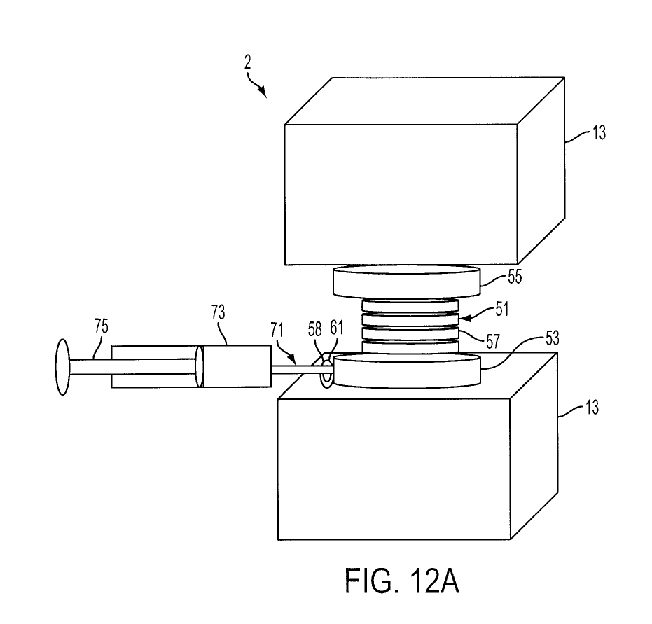

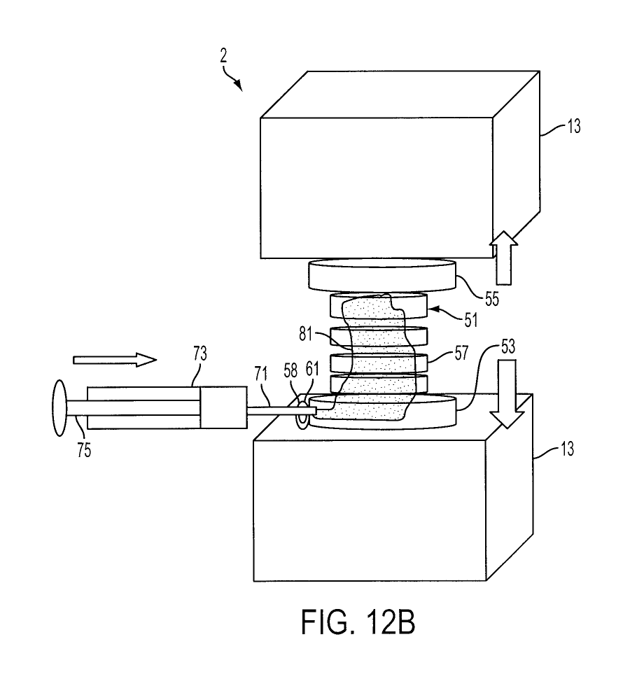

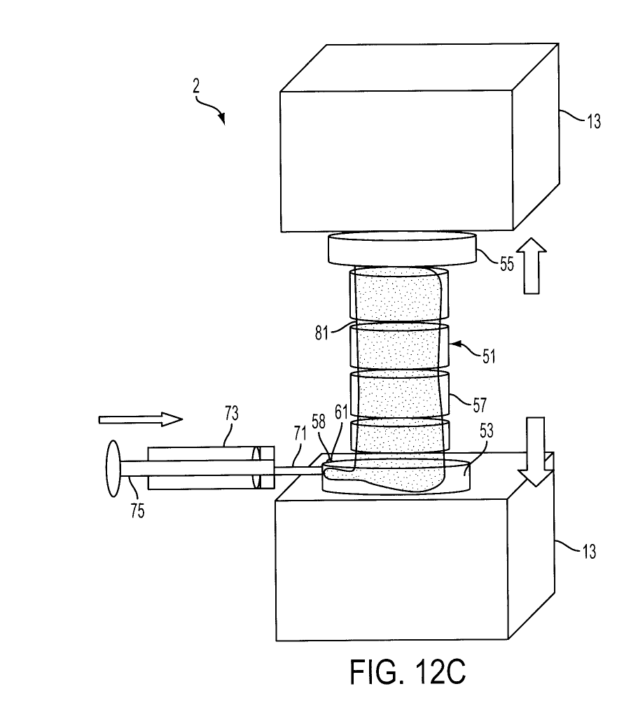

FIGS. 12A-C illustrate schematic side views of a portion of the spine 2 or spinal column with a portion removed there from. For instance, as illustrated, all of a vertebral body may be removed (or a portion of a vertebral body) providing two adjacent vertebrae 13. FIG. 12A illustrates the cage 51 placed and rotated accordingly by the driver element 71 into the spine 2 having utilized the present invention posterior approach while in a collapsed or non-deployed state. FIG. 12B illustrates the cage 51 in a partially expanded or deployed state using the driver element 71 and its related components. Some related components may be the driver mechanism 73 that is adapted to deliver the cement 81 or filler material into the cage 51. In an approach, the driver is adapted to deliver the cement or filler material under negative pressure. The driver mechanism 73 of the driver element 71 may comprise an actuator 75 such as a valve, regulator, manifold, syringe, flow-driver, pump, or piston, or any combination thereof, etc. to advance the cement or filler. For example, in an embodiment, the driver element comprises a tube that is connected to the cage and allows its placement. The driver may be locked or secured to the cage and a valve mechanism(s) is provided that prevents air from entering the cage to prevent air bubbles from forming in the cage body. Yet the valve mechanism(s) or the like also allows filler material to enter the cage under positive pressure. The driver element is 71 is in communication with the cage 51 (at the lower plate 53 in this instance) at a cage aperture 58. FIG. 12C illustrates the cage 51 in a fully expanded or deployed state using the driver element 71 and its related components.

Sill referring to FIGS. 12A-C (and any of the embodiments discussed throughout), the driver element 71 can be locked or secured using the cage aperture 58 as the locking mechanism. Alternatively, a separate locking or securing device, such as a driver lock 61, may also be utilized. It should be appreciated that the driver lock or securing means of the aperture may be a variety of locking means such as, but not limited thereto, a lock, pin, stop, stay, brace, latch, catch or latch, threading, etc. Indifferent if it's the aperture 58 or lock 61, by locking or securing of the driver element 71 the driver element 71 can manipulate the cage 51 during insertion, rotation and placement (i.e., orientation) without losing grip or control of the cage 51 as desired or required. Although not illustrated, it should be appreciated that the functions of the driver element 71 (filling the filler into the cage and orienting the cage) may be accomplished with separated members or instruments, rather than a single member or instrument as illustrated.

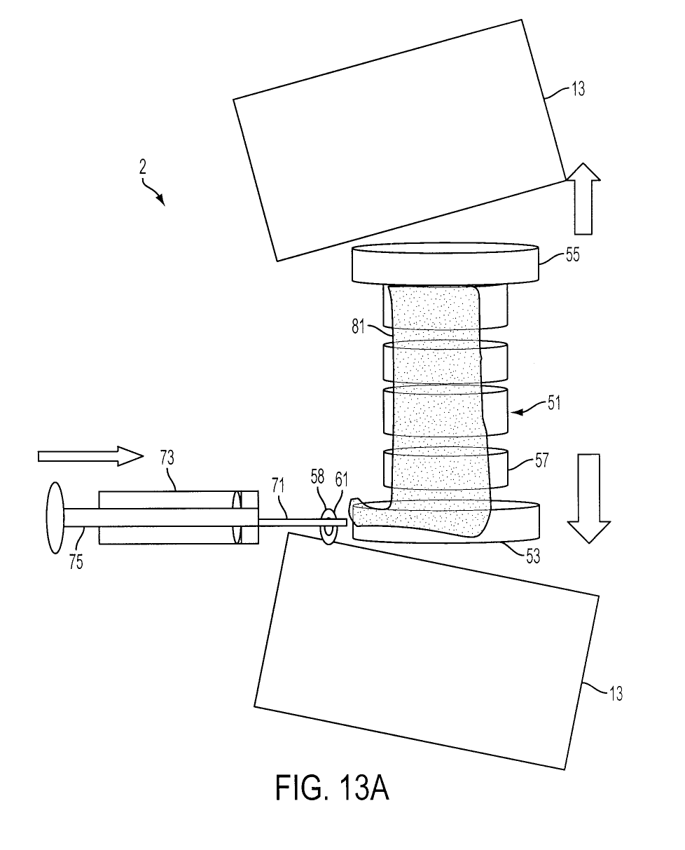

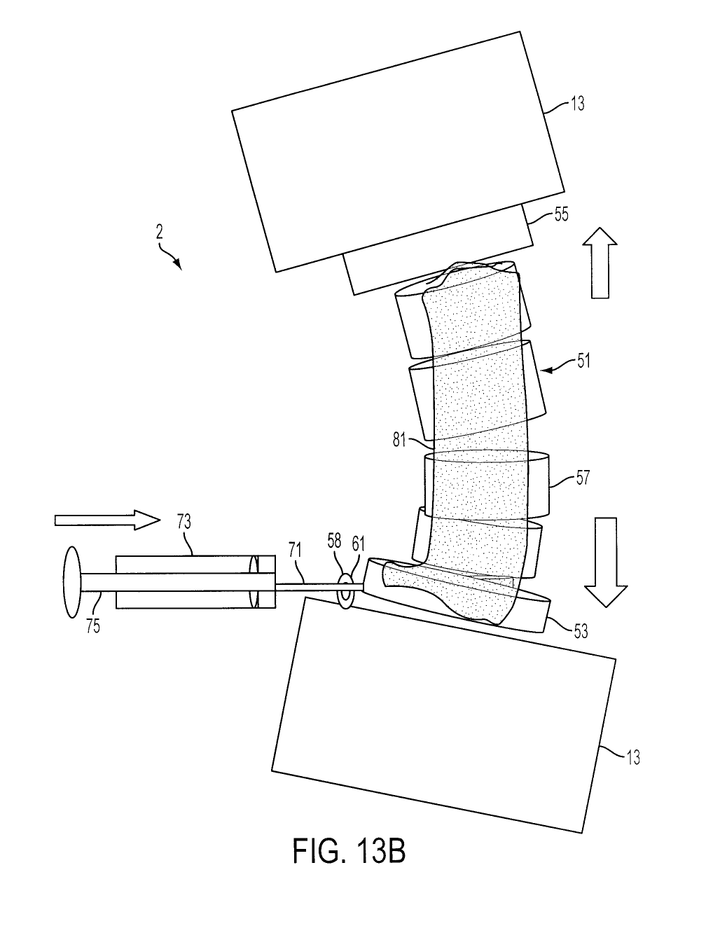

FIG. 13A schematically illustrates the cage 51 placed and rotated accordingly by the driver element 71 into the spine 2 that is diagnosed with kyphosis, and which the cage 51 is in a partially expanded or deployed state. FIG. 13B illustrates the cage 51 in a fully expanded or deployed state using the driver element 71 and its related components whereby the cage conforms to the contours or curvature of the spine diagnosed with kyphosis. It should be appreciated that because of the flexible nature of the cage, the cage and/or plates will match the local kyphosis lordosis of the spine segment to be reconstructed.

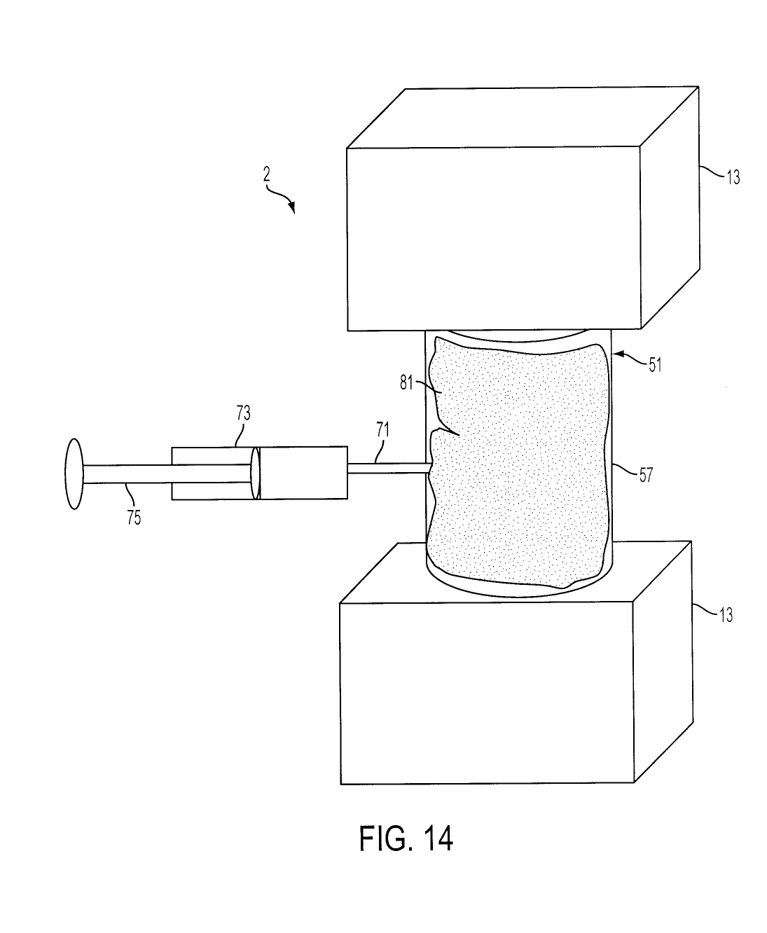

FIG. 14 schematically illustrates the cage 51 (without upper or lower plates) in a fully expanded or deployed state using the driver element 71 and its related components, and whereby the driver element is in communication with the cage body 57, rather than one or both of the upper or lower plates.

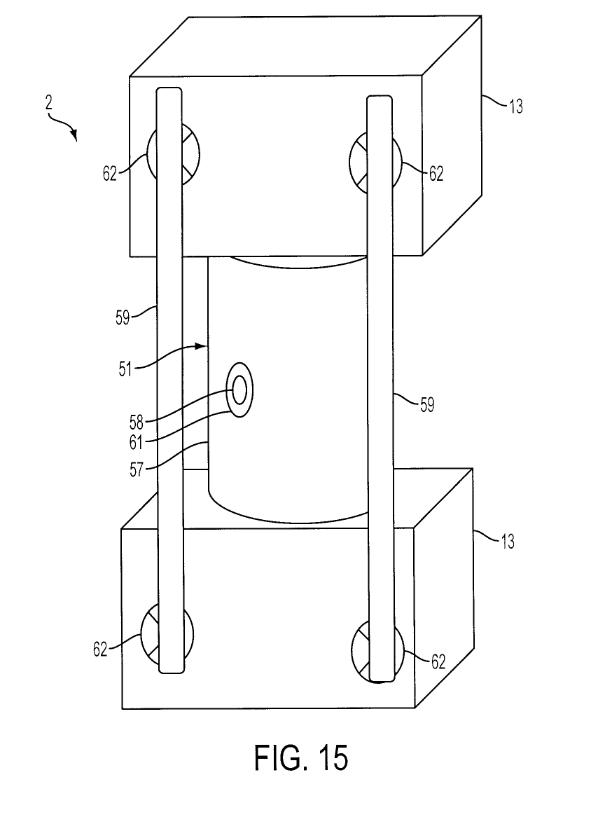

FIG. 15 schematically illustrates the cage 51 in a fully expanded or deployed state using the driver element 71 and its related components, and whereby cage rods 59 (such as titanium rods or other materials as desired or required) and pedicle screws 62 are implemented as part of the cage construction.

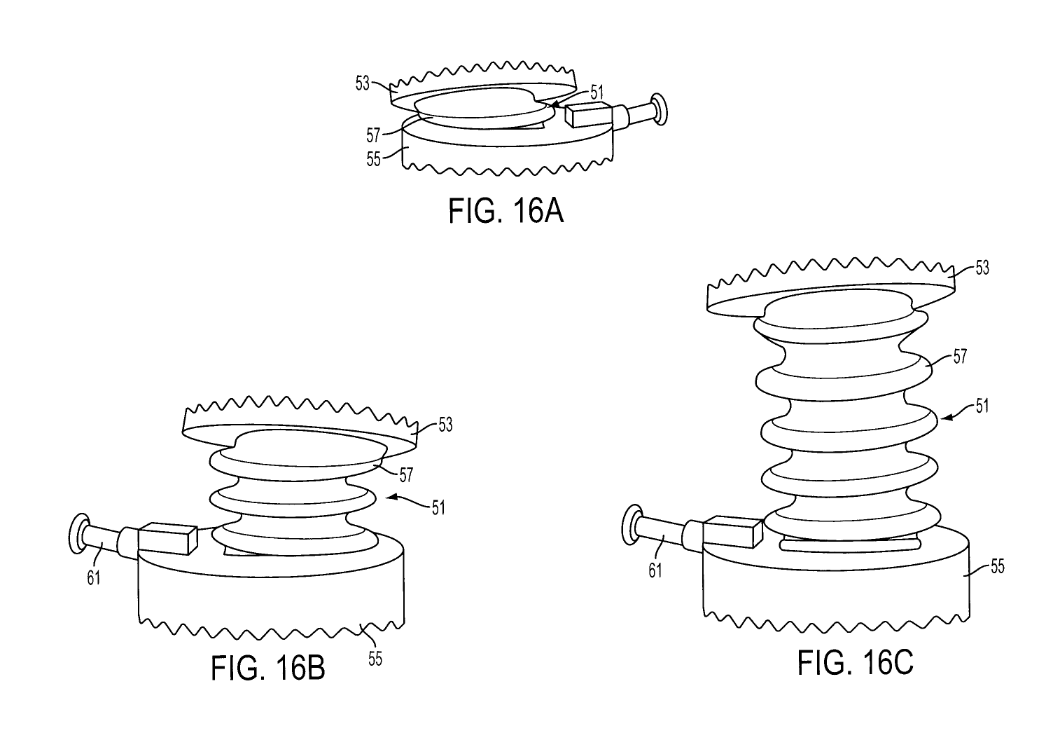

FIG. 16A schematically illustrates the cage 51 having a cage body 57 of the accordion type in the collapsed or non-deployed state. FIG. 16B illustrates the cage 51 of FIG. 16A while in the partially expanded or deployed state. FIG. 16C illustrates the cage 51 of FIG. 16A while in the fully expanded or deployed state. The number of the pleats/folds of the accordion/bellows, as well as the height/thickness of the pleats/folds of the accordion/bellows may be increased or decreased as desired or required.

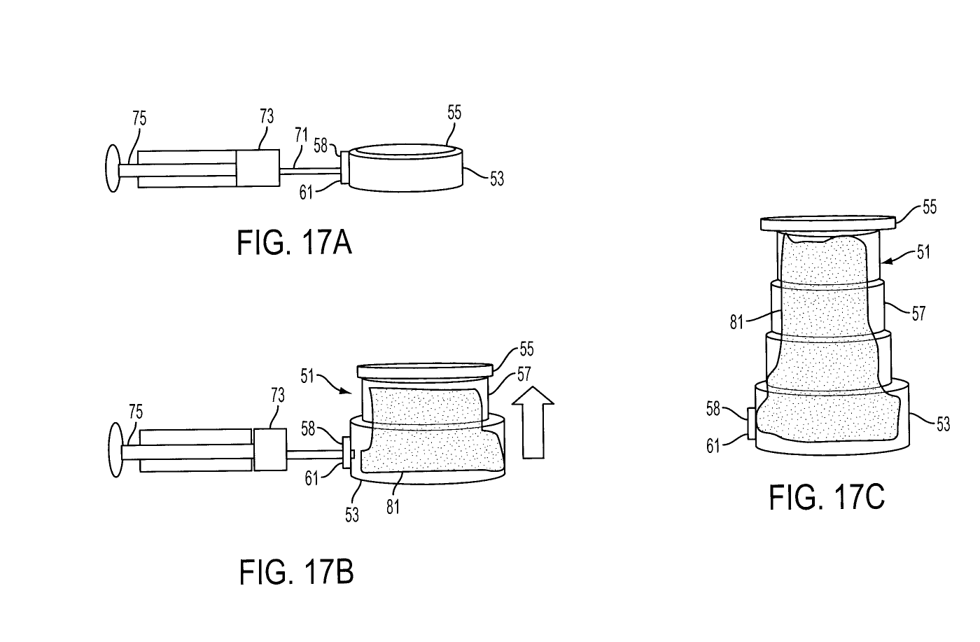

FIG. 17A schematically illustrates the cage 51 having a cage body 57 of the telescopic type (wedding cake) in the collapsed or non-deployed state. FIG. 17B illustrates the cage 51 of FIG. 17A in the partially expanded or deployed state. FIG. 17C illustrates the cage 51 of FIG. 17A in the fully expanded or deployed state.

An aspect of an embodiment of the present invention device and related method provides a cage to be inserted through a posterior approach. To accomplish such an objective, an expandable plastic tubing or an expandable series of cylinders may be implemented. For instance, a type of flexible tubing is an accordion or bellows type of tubing. The expandable cage have an extremely low profile structure to be inserted from the back. The present invention method and related method provides for introducing the cage from the back adjacent to the neural structures (i.e., spinal cord) and then rotate it 90 degrees, or as desired or required, to be able to expand it. In an exemplary embodiment the cage had to about the lateral cross section of the size of a face of a U.S. quarter coin and as little profile vertically (axially) as possible so it can be inserted from the back.

Regarding the design of the present invention cage filled with cement, a plastic cage or compatible biomaterial that will be filled with cement to expand the cage once its in its desired location, position and/or alignment.

It should be appreciated that in the case of an anterior approach or wide costotransveresectomy the cage can be inserted in a manner consistent with a mechanical cage without the need to rotate it in place, as the larger access allows its insertion without risk to the spinal cord. However, the present invention low profile cage with high expansion capabilities is adapted for a posterior and less invasive approach and is only feasible by rotating a low profile cage into the vertebrectomy defect.

It should be appreciated that various aspects of embodiments of the present device, method, system and materials may be implemented with the following devices, methods, systems and materials disclosed in the following U.S. Patent Applications, U.S. Patents, and PCT International Patent Applications that are hereby incorporated by reference herein:

1. U.S. Pat. No. 6,436,140, B1, Liu, et. al., “Expandable Interbody Fusion Cage and Method for Insertion”, Aug. 20, 2002.

2. U.S. Pat. No. 7,014,659, B2, Boyer, et. al., “Skeletal Reconstruction Cages”, Mar. 21, 2006.

3. U.S. Pat. No. 6,443,990, B1, Aebi, et. al., “Adjustable Intervertebral Implant”, Sep. 3, 2002.

4. U.S. Pat. No. 6,893,464, B2, Kiester, “Method and Apparatus for Providing an Expandable Spinal Fusion Cage”, May 17, 2005.

5. U.S. Pat. No. 5,665,122, Kambin, “Expandable Intervertebral Cage and Surgical Method”, Sep. 9, 1997.

6. U.S. Pat. No. 6,488,710, B2, Besselnik, “Reinforced Expandable Cage ad Method of Deploying”, Dec. 3, 2002.

7. U.S. Pat. No. 6,491,724, B1, Ferree, “Spinal Fusion Cage with Lordosis Correction”, Dec. 10, 2002.

8. U.S. Pat. No. 6,695,760, B1, Winkler, “Treatment of Spinal Metastases”, Feb. 24, 2004.

9. U.S. Pat. No. 5,236,460, Barber, “Vertebral Body Prosthesis”, Aug. 17, 1993.

10. U.S. Pat. No. 5,480,442, Bertagnoli, “Fixedly Adjustable Intervertebral Prosthesis”, Jan. 2, 1996.

11. U.S. Pat. No. 4,932,975, Main, et. al., “Vertebral Prosthesis”, Jun. 12, 1990.

12. U.S. Patent Application Publication No. US2005/0222681 B1, Richley, et. al., “Devices and Methods for Minimally Invasive Treatment of Degenerated Spinal Discs”, Oct. 6, 2005.

13. U.S. Patent Application Publication No. US2005/0283247 A1, Gordon, et. al., “Expandable Articulating Intervertebral Implant with Limited Articulation”, Dec. 22, 2005.

14. U.S. Patent Application Publication No. US2005/0283248 A1, Gordon, et. al., “Expandable Intervertebral Implant with Spacer”, Dec. 22, 2005.

15. U.S. Patent Application Publication No. US2006/0116767, A1, Magerl, et. al., “Implant Used in Procedures for Stiffening the Vertebral Column”, Jun. 1, 2006.

16. U.S. Patent Application Publication No. US2006/0129241, A1, Boyer, et. al., “Skeletal Reconstruction Cages”, Jun. 15, 2006.

17. U.S. Patent Application Publication No. US2006/0142858, A1, Colleran, et. al., “Expandable Implants for Spinal Disc Replacement”, Jun. 29, 2006.

18. U.S. Patent Application Publication No. US2002/0128716 A1, Cohen, et. al., “Spinal Surgical Prosthesis”, Sep. 12, 2002.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

I claim:

1. An intervertebral prosthesis device for posterior implantation between adjacent vertebrae of a subject, the device comprising:
a) a cage body, said cage body having a lower end and an upper end defining a vertical height there between;
said cage body having a transverse width and transverse length;
said cage body being flexible and expandable, wherein said cage body being adapted to:
change from a non-deployed state defining the vertical height being in a non-deployed vertical height (NVH) and expanded to a deployed state when filled with a filler material defining the vertical height being in a deployed vertical height (DVH), wherein said non-deployed vertical height cage body is less than or equal to the transverse height or transverse length of said cage body; and
b) a driver element in communication with said cage body adapted to:
insert said cage from the posterior of a subject and rotate said cage between the adjacent vertebrae, and
expand said cage body with the filler material.

2. The device of claim 1, wherein said non-deployed vertical height (NVH) cage body is less than or equal to each of the transverse height and transverse length of said cage body.

3. The device of claim 1, wherein said filler material comprises cement, polymethyl methacrylate (PMMA), biological resin, or bioactive paste, or any combination thereof.

4. The device of claim 1, wherein said expanding said cage body with the filler material comprises said driver element being in contact with said cage body to fill said filler material into said cage body to be used for expanding the cage body.

5. The device of claim 4, wherein said driver element comprises a driver mechanism adapted to move the filler material into said cage body.

6. The device of claim 5, wherein said movement of the filler material into said cage body is accomplished vacant of air forming bubbles in said cage body.

7. The device of claim 1, further comprising a lower endplate in communication with said lower end of said cage body.

8. The device of claim 7, wherein said expanding said cage body with the filler material comprises said driver element being in contact with said lower endplate to fill said filler material into said cage body to be used for expanding the cage body.

9. The device of claim 8, wherein said driver element comprises a driver mechanism adapted to move the filler material into said cage body.

10. The device of claim 9, wherein in said driver mechanism comprises at least one of: valve, regulator, manifold, syringe, flow-driver, pump, or piston.

11. The device of claim 9, wherein said cage body in said non-deployed state is under negative pressure.

12. The device of claim 11, wherein a valve is adapted to provide for said negative pressure.

13. The device of claim 12, wherein a valve is adapted to regulate filler material entering in and/or exiting from the cage body and regulate air from exiting from and/or entering in said cage body.

14. The device of claim 9, wherein said movement of the filler material into said cage body is accomplished vacant of air forming bubbles in said cage body.

15. The device of claim 14, wherein a valve is adapted to allow said filler material to enter said cage body without the formation of air forming bubbles in said cage body.

16. The device of claim 1, wherein said cage body comprises a flexible chamber.

17. The device of claim 16, wherein said flexible chamber comprises a tube, balloon, hose, cylinder, accordion-like structure, bellows, case, shell, enclosure, sleeve, or repository.

18. The device of claim 1, wherein said driver element is rotated about 90 degrees for orientation between the adjacent vertebrae.

19. The device of claim 1, wherein said driver element is rotated less than about 90 degrees for orientation between the adjacent vertebrae.

20. The device of claim 1, wherein said driver element is rotated greater than about 90 degrees for orientation between the adjacent vertebrae.

21. The device of claim 1, wherein said driver element is secured to said cage to allow for said insertion and rotation of said cage.

22. The device of claim 21, further comprising:
a locking mechanism, said locking mechanism adapted for said securing of said driver element to said cage.

23. A method for implanting an intervertebral prosthesis device between adjacent vertebrae from the posterior of a subject, sad method comprising:
inserting a cage body from the posterior of the subject into space defined between the adjacent vertebrae;
rotating said cage body between the adjacent vertebrae, and
expanding said cage body with the filler material to fill a space defined between the adjacent vertebrae.

24. The method of claim 23, wherein said filling into said cage body is accomplished vacant of air forming bubbles in said cage body.

25. The method of claim 23, wherein said filler material comprises cement, polymethyl methacrylate (PMMA), biological resin, or bioactive paste, or any combination thereof.

26. The method of claim 23, wherein:
said cage body having a lower end and an upper end defining a vertical height there between;
said cage body having a transverse width and transverse length;
said cage body being flexible and expandable, wherein said cage body being adapted to:
change from a non-deployed state defining the vertical height being in a non-deployed vertical height (NVH) and expanded to a deployed state during said filling with said filler defining the vertical height being in a deployed vertical height (DVH), wherein said non-deployed vertical height cage body is less than or equal to the transverse height or transverse length of said cage body.

27. The method of claim 26, wherein said non-deployed vertical height (DVH) said cage body is less than or equal to each of the transverse height and transverse length of said cage body.

28. The method of claim 23, wherein said cage body comprises a flexible chamber.

29. The device of claim 28, wherein said flexible chamber comprises a tube, balloon, hose, cylinder, accordion-like structure, bellows, case, shell, enclosure, sleeve, or repository.

30. The method of claim 23, wherein said rotation comprises rotating said cage body about 90 degrees for orientation between the adjacent vertebrae.

31. The method of claim 23, wherein said rotation comprises rotating said cage body less than about 90 degrees for orientation between the adjacent vertebrae.

32. The method of claim 23, wherein said rotation comprises rotating said cage body greater than about 90 degrees for orientation between the adjacent vertebrae.

33. The method of claim 23, further comprising:
securing said cage to allow for said insertion and said rotation.

34. The method of claim 33, further comprising:
providing a driver element; and
providing a locking mechanism for securing said driver element to said cage.

35. The method of claim 23, further comprising:
regulating said filler material entering in and/or exiting from said cage body, and
regulating air entering in and/or exiting from said cage body.
